(19) United States
(12) Patent Application Publication
(10) Pub. No.: US 2021/0142892 A1
(43) Pub. Date: May 13, 2021

(54) System and Method for Facilitating Short Term Staffing in the Healthcare Industry

(71) Applicant: Jeremy Jones, Cedar Park, TX (US)

(72) Inventor: Jeremy Jones, Cedar Park, TX (US)

(21) Appl. No.: 16/679,419

(22) Filed: Nov. 11, 2019

(51) Int. Cl. G16H 40/20; G06Q 10/10; G06Q 50/30

(52) U.S. Cl. CPC G16H 40/20; G06Q 50/30; G06Q 10/1053

(57) ABSTRACT

A system for obtaining staffing for a plurality of temporary staffing opportunities available at various healthcare facilities is disclosed. The system comprises a computing device in operable communication with a database to store information related to a plurality of candidates and a plurality of healthcare facilities. A calendar module receives scheduling information via the computing device and corresponds the scheduling information to a plurality of staffing availabilities. A hiring system transmits the plurality of staffing availabilities to the plurality of candidates, and a comparator compares data pertaining to the plurality of candidates with the plurality of staffing availabilities to match one or more of the plurality of candidates with one or more of the plurality of staffing availabilities.

### TECHNICAL FIELD

The embodiments generally relate to electronic services and more specifically relate to a system for facilitating staffing of a labor force via a third-party job placement service.

### BACKGROUND

The healthcare industry often suffers from understaffing, causing a deficit in the labor force. It can prove difficult for healthcare providers to source and adequately recruit quality candidates for various job openings. The American Hospital Association projects that the rate of understaffing will continue to rise in the coming years.

The growing demand for qualified healthcare professionals in the travel nurse industry requires healthcare facilities to rely on travel nurse agencies to source and staff open positions from a limited pool of healthcare professionals. To compound the problem, many of the healthcare professionals are seeking open positions in a specialized field of the healthcare industry.

There are currently no convenient resources for facilitating the recruitment and staffing of travel healthcare professions (such as travel nurses). This results in the personnel at the healthcare facility being forced to source and staff travel healthcare professionals using various staffing agencies, online resources, or by vetting applicants in-house. This additional effort results in a loss of time and economic resources, which in turn causes a multitude of downstream problems throughout the healthcare industry.

### SUMMARY OF THE INVENTION

This summary is provided to introduce a variety of concepts in a simplified form that is further disclosed in the detailed description of the embodiments. This summary is not intended to identify key or essential inventive concepts of the claimed subject matter, nor is it intended for determining the scope of the claimed subject matter.

The embodiments disclosed herein relate to a system for obtaining staffing for a plurality of temporary staffing opportunities available at various healthcare facilities. The system comprises a computing device in operable communication with a database to store information related to a plurality of candidates and a plurality of healthcare facilities. A calendar module receives scheduling information via the computing device and corresponds the scheduling information to a plurality of staffing availabilities. A hiring system transmits the plurality of staffing availabilities to the plurality of candidates, and a comparator compares data pertaining to the plurality of candidates with the plurality of staffing availabilities to match one or more of the plurality of candidates with one or more of the plurality of staffing availabilities.

In one aspect, a verification module verifies one or more credentials provided by the plurality of candidates. The one or more credentials are comprised of one or more of the following: a vaccination history, one or more licenses, and one or more qualifications.

In one aspect, an employment history is stored in the database which may be viewed by personnel at the healthcare facility to determine a suitable hirable candidate.

In one aspect, an evaluation module receives one or more evaluations and correspond the evaluation to one of the plurality of healthcare professionals. Evaluations may be provided by a healthcare facility, by candidates, or by other users of the system.

In one aspect, a calendar module indicates one or more employable candidates to a user at the healthcare facility.

In one aspect, the scheduling information comprises one or more of the following: one or more dates available, one or more days of the week available, one or more locations, a travel distance, and one or more times available.

In one aspect, a communication module provides a communication means between the plurality of healthcare facilities and the plurality of candidates.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1 illustrates a block diagram of the network infrastructure, according to some embodiments;

FIG. 2 illustrates a block diagram of the network infrastructure, according to some embodiments;

FIG. 3 illustrates a block diagram of the server engine and associated modules, according to some embodiments;

FIG. 4 illustrates a flowchart of a use case diagram, according to some embodiments;

FIG. 5 illustrates a block diagram of a candidate credential table, according to some embodiments;

FIG. 6 illustrates a view of the offer generation system, according to some embodiments; and

FIG. 7 illustrates a flowchart of a method for staffing temporary position, according to some embodiments.

### DETAILED DESCRIPTION

The specific details of the single embodiment or variety of embodiments described herein are to the described system and methods of use. Any specific details of the embodiments are used for demonstrative purposes only, and no unnecessary limitations or inferences are to be understood therefrom.

Before describing in detail exemplary embodiments, it is noted that the embodiments reside primarily in combinations of components and procedures related to the system. Accordingly, the system components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

In general, the embodiments described herein provide systems and methods for registering, staffing, and evaluating a workforce for a facility. The embodiments may be used to recruit candidates, skilled laborers, and/or unskilled laborers to fill temporary and/or permanent positions available at a facility, such as a healthcare facility. More specifically, candidates may be provided with access to more employment opportunities in various locations during pre-specified periods of time. The system may allow for performance-based pay and control over a work schedule. At the same time, hiring facilities, such as healthcare facilities may gain the benefit of accessing a larger pool of potential candidates from which they may review candidate credentials, candidate schedules, and contact the candidate. As a result, the system greatly improves hiring efficiency while improving the overall candidate quality.

As used herein, the term “user(s)” may refer to candidates who are seeking staffing opportunities at various facilities, hiring managers at the facilities, representatives of the facilities, administrative users, or other professionals involved in the hiring process. In a particular example, the candidate is a healthcare professional (such as a nurse, travel nurse, or other profession in the healthcare setting). The system may be particularly useful to healthcare professionals who frequently travel to various locations and are employed at each location temporarily, such as travel nurses. To participate in the staffing marketplace, users may register with the system. The registration process may involve several steps, and in some embodiments, may involve taking screening tests, answering surveys, providing references, confirming employment and/or education history, confirming licensure status, providing documents, and/or executing contracts. Some of the registration steps may be performed using the system, and some may be performed manually using paper documents, mail and/or in person interviews.

FIG. 1 illustrates a computer system 100, which may be utilized to execute the processes described herein. The computer system 100 is comprised of a standalone computer or mobile computing device, a mainframe computer system, a workstation, a network computer, a desktop computer, a laptop, or the like. The computer system 100 includes one or more processors 110 coupled to a memory 120 via an input/output (I/O) interface. Computer system 100 may further include a network interface to communicate with the network 130. One or more input/output (I/O) devices 140, such as video device(s) (e.g., a camera), audio device(s), and display(s) are in operable communication with the computer system 100. In some embodiments, similar I/O devices 140 may be separate from computer system 100 and may interact with one or more nodes of the computer system 100 through a wired or wireless connection, such as over a network interface.

Processors 110 suitable for the execution of a computer program include both general and special purpose microprocessors and any one or more processors of any digital computing device. The processor 110 will receive instructions and data from a read-only memory or a random-access memory or both. The essential elements of a computing device are a processor for performing actions in accordance with instructions and one or more memory devices for storing instructions and data. Generally, a computing device will also include, or be operatively coupled to receive data from or transfer data to, or both, one or more mass storage devices for storing data, e.g., magnetic, magneto-optical disks, or optical disks; however, a computing device need not have such devices. Moreover, a computing device can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive).

A network interface may be configured to allow data to be exchanged between the computer system 100 and other devices attached to a network 130, such as other computer systems, or between nodes of the computer system 100. In various embodiments, the network interface may support communication via wired or wireless general data networks, such as any suitable type of Ethernet network, for example, via telecommunications/telephony networks such as analog voice networks or digital fiber communications networks, via storage area networks such as Fiber Channel SANs, or via any other suitable type of network and/or protocol.

The memory 120 may include application instructions 150, configured to implement certain embodiments described herein, and a database 160, comprising various data accessible by the application instructions 150. In one embodiment, the application instructions 150 may include software elements corresponding to one or more of the various embodiments described herein. For example, application instructions 150 may be implemented in various embodiments using any desired programming language, scripting language, or combination of programming languages and/or scripting languages (e.g., C, C++, C#, JAVA®, JAVASCRIPT®, PERL®, etc.).

The steps and actions of the computer system 100 described in connection with the embodiments disclosed herein may be embodied directly in hardware, in a software module executed by a processor, or in a combination of the two. A software module may reside in RAM, flash memory, ROM memory, EPROM memory, EEPROM memory, registers, a hard disk, a removable disk, a CD-ROM, or any other form of storage medium known in the art. An exemplary storage medium may be coupled to the processor 110 such that the processor 110 can read information from, and write information to, the storage medium. In the alternative, the storage medium may be integrated into the processor 110. Further, in some embodiments, the processor 110 and the storage medium may reside in an Application Specific Integrated Circuit (ASIC). In the alternative, the processor and the storage medium may reside as discrete components in a computing device. Additionally, in some embodiments, the events or actions of a method or algorithm may reside as one or any combination or set of codes and instructions on a machine-readable medium or computer-readable medium, which may be incorporated into a computer program product.

Also, any connection may be associated with a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. “Disk” and “disc,” as used herein, include compact disc (CD), laser disc, optical disc, digital versatile disc (DVD), floppy disk and Blu-ray disc where disks usually reproduce data magnetically, while discs usually reproduce data optically with lasers. Combinations of the above should also be included within the scope of computer-readable media.

In some embodiments, the system is world-wide-web (www) based, and the network server is a web server delivering HTML, XML, etc., web pages to the computing devices. In other embodiments, a client-server architecture may be implemented, in which a network server executes enterprise and custom software, exchanging data with custom client applications running on the computing device.

In reference to FIG. 2, a schematic of the system is disclosed, comprising a community of users 200 which include a plurality of candidates 210 in communication with a candidate-associated computing device 220, and a plurality of hiring professionals associated with a hiring facility 225 (such as a healthcare facility) in communication with a hiring facility computing device 230. Each user in the community is in communication with the system and other users via the network 130. The facility 225 may include a secure network 240 to allow secure communications to and from the facility 225 as well as communications within the facility 225.

In one embodiment, the candidate 210 may be a health care worker, such as a Nurse (e.g. Nurse Practitioner (“NP”), Registered Nurse (“RN”), Licensed Practical Nurse (“LPN”), Licensed Vocational Nurse (“LVN”), Certified Nurse's Assistant (“CNA”), and/or a Health Care Aide (“HCA”)). In such cases, the hiring facility 225 may be a health care provider such as a hospital, nursing home, or other facility, or a private residence. A hiring facility 225 may authorize an individual to act as a manager who can perform administrative tasks within the system on behalf of the hiring facility 225.

FIG. 3 illustrates a server engine 300 comprised of a plurality of modules operable to perform the plurality of tasks described by the various embodiments. A registration module 310 transmits user registration information and categorizes the information. Registration information may include user contact information, user-type (e.g., candidate, hiring personnel, facility representative, administrator, etc.), user login credentials, usernames, passwords, and other information known in the arts. The registration module 310 may also operate to collect and categorize user credentials such as work history, resumes, licenses, experience, and other credentials, especially those common in regulated skilled labor industries. A verification module 320 operates to verify credentials input by the user. Credentials may include resumes, certifications, licenses, professional experience, professional references, degrees, and other professional credentials known in the arts. Further the verification module 320 may verify a vaccine history of the user, which may be especially useful for users working in the healthcare industry. A scheduling module 330 allows the candidate to input availability information, such as dates, times, and locations, for which they are available to work, which will be matched by a comparator 340 to staffing availabilities analyzed by a calendar module which receives input from users at the facilities.

In some embodiments, credentials may include license information and/or certification information which is requested or received by the system. The information may include location and type of license/credential, when the license/certificate was issued, the time period for which the license/certificate is valid, and an expiration date thereof if applicable.

Credential information may include such items as the type of credential or skill, a subcategory of a specialized skill within the selected broad category, a short description of the skill, the number of years of experience that the worker has with that skill, and the years since the worker's most recent use of the skill (e.g., currently using this skill, in the past 12 months, in the last 3 years, over 3 years ago). Certificates information may include, for example, for a nurse, an expiration date of a nursing license, an expiration date of a CPR certification, an expiration date of a TB immunization, an expiration date of a current physical and/or the date of Hepatitis B Immunization.

In some embodiments, the availability information may include dates, days of the week, times that the worker is available or willing to work, a preferred geographic location, and/or a minimum travel distance. In some instances, the method also includes collecting registration information from the worker, such as a name, email address, phone number, licensure information, union membership information, skills, previous work history, and similar information. In some cases, the registration information may include a preferred method of communication (e.g., email, cell phone). In some implementations, the matching process may also be based on the workers' skills, skill level, number of years using the skill, and/or how recently the skill was used.

In some embodiments, the workers may be screened prior to being considered for the matching process. Screening may be performed by the verification module 320. The screening may include the administration of one or more tests aimed at gauging the worker's knowledge of a particular subject area or skill. In the travel nursing context, as just a few of the many possible examples, the tests may require the workers to identify certain drug interactions, recite the process for administering an intravenous drip, or calculate dosages.

FIG. 4 illustrates a method for obtaining staffing for a plurality of staffing opportunities available at various healthcare facilities. In step 400, scheduling information is received from a plurality of candidates and stored in the database to permit the system to analyze the data as described hereinabove. In step 410, a comparator compares scheduling availabilities with scheduling information provided by the candidates. The comparator may compare preferred date availabilities, time availabilities, locations, and other known parameters to determine one or more appropriate candidates in step 420. In step 430, the system may validate, via the validation module, credentials submitted by the candidate. In some cases, the validation module may require the candidate to perform an exam, submit documents, submit references, or other processes which validate the experience and knowledge of the candidate in optional step 440.

FIG. 5 illustrates schematic of the candidate credential table 500 having data stored in the database and organized by the verification module to display candidates and their associated credentials. The candidate profile 510 comprises data related to the job seeker, such as experience, education, tasks performed, skills possessed, scheduling information, etc.

FIG. 6 illustrates an offer generation system 600 having an offer generation interface 610. The offer generation interface 610 permits the system to generate an offer for employments including a plurality of employment terms 620, e.g., start date, duration of employment (if applicable), wages (i.e., salary or hourly rate), and other employment terms known in the arts. The verification module may then verify each credential with documents uploaded to the database.

FIG. 7 illustrates a flowchart of a method for staffing a temporary position at a facility. In step 700, the candidate submits a candidate profile (see FIG. 5) which is stored in the database. A user at the facility may search, or otherwise review a plurality of candidate profiles to determine a suitable candidate. In some embodiments, the system may automatically present the user at the facility with candidates who meet predefined parameters. In step 710, one or more suitable candidates are accepted and contacted via the communication module. In step 720, the verification module verifies the candidate's credentials. In step 730, the communication module transmits the user's credentials to the user at the facility and permits the user at the facility to review the user's credentials. In optional step 740, the user at the facility transmits an invitation to interview with one or more person(s) at the facility, which has verified and contacted the candidate. In step 750, the user at the facility determines if the candidate is suitable and selects an accept, deny, and/or a further inquiry option to either accept the candidate for an available position, deny the candidate for an available position, or request another interview, request more information, or request another interaction from the candidate.

Many different embodiments have been disclosed herein, in connection with the above description and the drawings. It will be understood that it would be unduly repetitious and obfuscating to describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

An equivalent substitution of two or more elements can be made for anyone of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a subcombination or variation of a subcombination.

It will be appreciated by persons skilled in the art that the present embodiment is not limited to what has been particularly shown and described hereinabove. A variety of modifications and variations are possible in light of the above teachings without departing from the following claims.

What is claimed is:

1. A system for obtaining staffing for a plurality of staffing opportunities available at various healthcare facilities, the system comprising:
a computing device in operable communication with a database to store information related to a plurality of candidates and a plurality of healthcare facilities;
a calendar module to receive scheduling information via the computing device, the scheduling information corresponding to a plurality of staffing availabilities provided by one or more healthcare professionals;
a hiring system configured to transmit the plurality of staffing availabilities to the plurality of candidates;
a comparator to compare data pertaining to the plurality of candidates with the plurality of staffing availabilities to match one or more of the plurality of candidates with one or more of the plurality of staffing availabilities.

2. The system of claim 1, further comprising an evaluation module configured to receive one or more evaluations and correspond the evaluation to one of the plurality of healthcare professionals.

3. The system of claim 2, wherein the one or more evaluations are displayed on the computing device.

4. The system of claim 1, further comprising a calendar module to indicate one or more employable healthcare professionals.

5. The system of claim 1, wherein the scheduling information comprises one or more of the following: one or more dates available, one or more days of the week available, one or more locations, a travel distance, and one or more times available.

6. A system for obtaining staffing for a plurality of staffing opportunities available at various healthcare facilities, the system comprising:
a computing device in operable communication with a database to store information related to a plurality of candidates and a plurality of healthcare facilities;
a calendar module to receive scheduling information via the computing device, the scheduling information corresponding to a plurality of staffing availabilities provided by one or more healthcare professionals;
a hiring system configured to transmit the plurality of staffing availabilities to the plurality of candidates;
a comparator to compare data pertaining to the plurality of candidates with the plurality of staffing availabilities to match one or more of the plurality of candidates with one or more of the plurality of staffing availabilities
a verification module to verify one or more credentials provided by the plurality of candidates.

7. The system of claim 6, wherein the one or more credentials are comprised of one or more of the following: a vaccination history, one or more licenses, and one or more qualifications.

8. The system of claim 7, further comprising an employment history stored in the database, wherein the employment history viewable via the computing device.

9. The system of claim 6, further comprising an evaluation module configured to receive one or more evaluations and correspond the evaluation to one of the plurality of healthcare professionals.

10. The system of claim 9, wherein the one or more evaluations are displayed on the computing device.

11. The system of claim 6, further comprising a calendar module to indicate one or more employable candidates.

12. The system of claim 6, wherein the scheduling information comprises one or more of the following: one or more dates available, one or more days of the week available, one or more locations, a travel distance, and one or more times available.

13. The system of claim 6, further comprising a communication module to provide a communication means between the plurality of healthcare facilities and the plurality of candidates.

14. A method for obtaining staffing for a plurality of staffing opportunities available at various healthcare facilities, the method comprising the steps of:
receiving a plurality of scheduling information from a plurality of candidates;
storing the plurality of staffing availabilities in a database in operable communication with a computing device;
comparing, via a comparator in operable communication with the database via a server, the plurality of staffing availabilities with scheduling information provided by the plurality of candidates;
determining, via a scheduling module, appropriate healthcare professionals to correspond to the plurality of scheduling information to determine one or more employable candidates.

15. The method of claim 14, wherein the one or more credentials are comprised of one or more of the following: a vaccination history, one or more licenses, and one or more qualifications.

16. The method of claim 14, further comprising an employment history stored in the database, wherein the employment history viewable via the computing device.

17. The system of claim 16, further comprising an evaluation module configured to receive one or more evaluations and correspond the evaluation to one of the plurality of candidates.

18. The method of claim 17, wherein the one or more evaluations are displayed on the computing device.

19. The system of claim 14, further comprising a calendar module to indicate one or more employable candidates.

20. The method of claim 14, wherein the scheduling information comprises one or more of the following: one or more dates available, one or more days of the week available, one or more locations, a travel distance, and one or more times available.
